(19) United States
(12) Patent Application Publication
(10) Pub. No.: US 2010/0048577 A1
(43) Pub. Date: Feb. 25, 2010

(54) Method for treating benign prostate hyperplasia using resveratrol

(76) Inventors: Joerg R. Leheste, Rocky Point, NY (US); German Torres, Sea-Cliff, NY (US)

Correspondence Address: FULBRIGHT & JAWORSKI, LLP, 666 FIFTH AVE, NEW YORK, NY, 10103-3198, US

(21) Appl. No.: 12/583,484

(22) Filed: Aug. 21, 2009

Related U.S. Application Data
(60) Provisional application No. 61/189,760, filed on Aug. 22, 2008.

(51) Int. Cl. A61K 31/05; A61K 31/496; A61K 31/498; A61K 31/429

(52) U.S. Cl. 514/249; 514/733; 514/254.02; 514/368

(57) ABSTRACT

The invention relates to the discovery that resveratrol, and substituted forms of resveratrol, are effective in alleviating benign prostate hyperplasia. Formulations containing the resveratrol or resveratrol derivative, lack omega-3 fatty acids, and also contain no other compounds which activate SIRT1 and PGCα.

### RELATED APPLICATION

This application claims priority of provisional application 61/189,760, filed Aug. 22, 2008, and incorporated by reference in its entirety.

### FIELD OF THE INVENTION

This invention relates to methods for treating or for preventing benign prostate hyperplasia, or “BPH.” More specifically, it relates to methodologies as described, where a patient is administered an effective amount of “resveratrol.”

### BACKGROUND OF THE INVENTION

BPH is a condition which affects the prostate gland in men. In brief, the prostate gland is found between the bladder and the urethra. As men age, their prostate glands enlarge. This, in turn, results in compression of in the urethra, with concomitant lessening of urine flow.

The symptoms of BPH are well known and need not be reiterated here. These symptoms are shared by other conditions, so medical testing is necessary to diagnose BPH correctly. When BPH is in fact diagnosed, there are several current therapeutic options.

Surgery is one venue for alleviation of the condition; however, the risks and inconvenience of surgery are well known.

Drug treatments are also available. Perhaps the best known of these is Finasteride, which is also sold under the brand name “Proscar,” and dutasteride, also sold as “Adovart.” Both drugs function by blocking production of a hormone which causes the prostate to enlarge. Neither drug has been shown to be universally helpful.

Alpha blockers have also been used to alleviate the symptoms of BPH; however, they do not reduce prostate size.

Saw palmetto has also been used to treat BPH, with some success; however, it is clear that additional therapies are desirable and necessary.

Resveratrol, a stilbene, also referred to as trans-3,5,4′-trihydroxystilbene, 3,4′,5-stilbenetriol, trans-Resveratrol, and (E)-5-(p-Hydroxystyryl resorcinol) has the following structure:

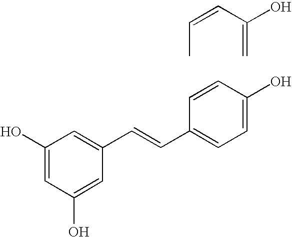

It is naturally produced by plants, especially grapes. It is also found in blueberries, and mulberries. Various therapeutic uses have been reported for it, including anti-inflammatory anti-cancer and blood sugar lowering effects.

Reports from animal studies involving the use of resveratrol in cancer treatment are extensive; however, BPH is caused by a mechanism which differs from the mechanisms of oncogenesis, and there are no reports in the scientific literature, of which the present inventions are aware involving use of resveratrol in treating BPH.

The patent literature describes compositions for treating BPH where resveratrol is used as an adjunct to an active, omega-3 fatty acid. See in this regard, issued U.S. Pat. Nos. 6,656,969 and 6,680,342, as well as published patent applications 2003/0054053; 2003/0054357; 2003/0055108; 2003/0055714; 2005/0070612; and 2007/0105931, all to Young, et al., all of which are incorporated by reference herein. Published patent application 2006/0069151 to Barella, et al., also incorporated by reference, teaches lycopene as an active ingredient for treating BPH, with resveratrol as a potential additive, while van Helvoort, et al., published U.S. patent application 2004/0259815, incorporated by reference, teaches a 3-part formulation where two inhibitors of the G1/S phase of the cell cycle, two or more inhibitors of the G2/M phase of the cell cycle, and two or more inhibitors of tyrosine kinase activity are combined. The second component can be a flavolignan, such as a hydroxylated stilbene, including resveratrol.

In all of these formulations, resveratrol is not described as the active ingredient; rather, as indicated supra it is used as an additive to the formulations containing, e.g., an omega-3 fatty acid, or some other molecule, which activates SIRT1 and PGC-α.

It has now been found, surprisingly, that resveratrol, by itself, i.e., in the absence of omega-3 fatty acids or SIRT1 and PGC-α activators, has a therapeutically positive effect on the treatment and prevention of BPH.

The details of this invention are set forth in the disclosure which follows.

### DETAILED DESCRIPTION OF PREFERRED EMBODIMENTS

### Example 1

Sexually mature Sprague Dawley rats were given two, intramuscular injections of testosterone enanthate, at 25 mg. This induced BPH.

Animals were then divided into different groups. A first group of 5 animals received finasteride, via subcutaneous injection at a dose of 10 mg. A second group received 1 g/kg of body weight of resveratrol, mixed into their diet. A third group received saw palmetto, at 625 mg/kg, mixed into their diet. The control group received the vehicle for the active material only with no active ingredient.

Following 29 days of treatment, all animals were sacrificed. Prostates were dissected free of fat, and weighed.

The injection of the testosterone enanthate increased the prostate weight/body weight ratio by about 2-fold. Analysis of hemotoxylin and eosin stained sections showed an increase in stromal and epithelial cell numbers, without changes in cell morphology.

Finasteride completely reversed the effect, which was expected, while no change was seen in the animals which received saw palmetto. Resveratrol reversed the induced BPH in a matter strikingly similar to that of finasteride.

### Example 2

These experiments were designed to determine whether resveratrol and trimethoxystilbene could cure or prevent BPH.

Experimental animals received diets that were fortified with one of trans-resveratrol (1 g/kg), trimethoxystilbene (1 g/kg), or saw palmetto (625 mg/kg). An additional group of animals received injections of finasteride fourteen days after the start of the experiment. The animals received one, subcutaneous injection of 10 mg of finasteride in 0.1 ml dimethyl sulfoxide on 5 consecutive days. All animals, except the absolute control group, received injections of testosterone enanthate (25 mg, in 0.1 ml mineral oil). The injections were administered intramuscularly, on days 1 and 7 of the study. The absolute control group received intramuscular injections of 0.1 ml of mineral oil.

At day 29, all test animals were weighed, sacrificed and their prostates were dissected free of fat and weighed. Prostate weight/body weight indices were determined for each group.

In summary, the results showed that the prostates of animals which received injections of testosterone enanthate were approximately 2 fold heavier than controls. The animals which received finasteride, resveratrol, or trimethoxystilbene for 28 days had prostates indistinguishable from controls, while animals which received saw palmetto, had enlarged prostates.

### Example 3

It had been observed that, even after prostates had been enlarged by injections with testosterone enanthate, resveratrol was able to reduce prostate weight. Experiments were designed to try to determine the mechanism for this.

Normal human prostate endothelial cells were obtained from the ATCC (CRL-11611), and were either treated or not treated with resveratrol, at 50 μM, for 48 hours. Gene expression of the cells was compared, via standard methods.

Of the 376 genes known to be involved with apoptosis, about 10% showed significant down regulation following treatment. The majority of these genes, and their expression products, are known to be responsible for cell cycle progression and increased proliferation such as CDK4, CSE1L, and MAP2K1, elevated energy metabolism (GPX1), and protein syntheses (RPL3, RPS10). Collectively these findings suggest that decreased mitosis, and not increased apoptosis, is the driving force for the reduction of prostate weight.

### Example 4

In additional experiments following the details of example 2, supra, levels of PSA expression were tested.

Prostate tissue from the experimental animals was subjected to quantitative PCR, using well known methods. The expression of PSA was compared in untreated, and in resveratrol, and trimethoxystilbene treated animals.

The animals which received resveratrol or trimethoxystilbene had a level of expression that was about ⅓ of that of the controls.

The foregoing sets forth the features of the invention which is a method for treating or preventing BPH in a subject, comprising administering to said subject an amount of an omega-3 fatty acid free composition which consists essentially of resveratrol, and contains no other compounds which activate SIRT1 and PGC-α, wherein said resveratrol containing composition is administered in an amount sufficient to treat or to prevent BPH. Formulations consisting essentially of resveratrol may also be administered in accordance with the invention.

The formulations which contain resveratrol may be administered, e.g., buccally, per os, intravenously, intramuscularly in the form of nanoparticles to extend half life, sublingually, rectally via, e.g., suppository or via any means that is most expedient for the subject, which is preferably a human being. Oral administration via, e.g., a dietary supplement, is especially preferred. The resveratrol is preferably administered in the form of tablets, liquids, capsules, or any other medium that is expedient for oral use. The dose administered will vary, depending upon the individual patient and severity of the condition; however, a dose of from about 10 mg-10 g per day, preferably 25 mg-7 g per day, and most preferably about 50 mg-5 g per day, is contemplated for mammals, especially humans. Such doses are preferred for all of the aforementioned modes of administration. It is anticipated that each of these modulaties will result in a minimum serum level of resveratrol of 10 ng/ml.

The active ingredient may also be administered probiotically, via enteric bacteria (e.g., Lactobacillus sp, Enterococcus sp, etc.), transformed or transfected with enzymes which produce resveratrol.

Probiotic administration such that a stable level of at least 10 ng/ml of trans resveratrol in serum is contemplated. Determining the amount of bacteria necessary to achieve this is within the ambit of the skilled artisan.

Further, transdermal and transepidermal patches may be used to administer the trans resveratrol, so as to achieve the aforementioned serum levels.

By “resveratrol,” it is to be understood that the compound as set forth supra, as well as pharmaceutically acceptable salts, are preferred. These can be, e.g., salts which are designed to be more soluble in an aqueous environment than resveratrol itself, as well as salts that are designed to release active ingredients more slowly, and over a longer period of time, than is resveratrol per se.

Also included are forms of resveratrol where one or more of its three —OH groups is substituted by an alkyl, alkenyl, or alkynyl group, which may be straight chained, or branched or contain a ring structure, which itself may be substituted by other molecules. The substituents may contain from 1 to 20, preferably 1 to 10, and most preferably from 1-6 carbon atoms. One, two, or all 3 hydroxyl groups may be so substituted. Exemplary, but not limiting examples, are 3,5,4′-trimethoxystibene, and 3,5′-dimethoxy-4-stilbenol. Related compounds, such as those disclosed in Mine, et al., Nature, 450:712-716 (2007), incorporated by reference, are also encompassed by the invention. These compounds are:

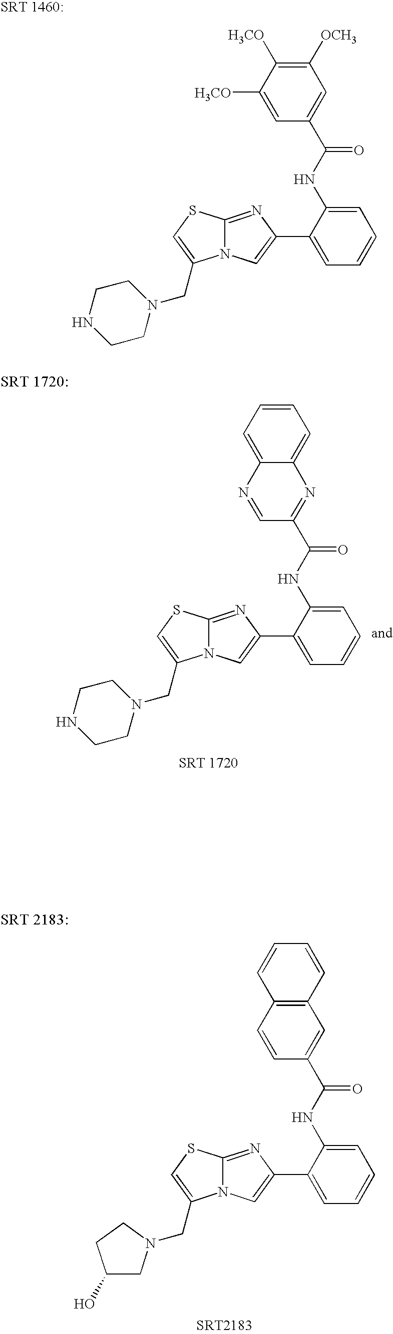

Other features of the invention will be clear to the skilled artisan, and need not be referenced here.

The terms and expression which have been employed are used as terms of description and not of limitation, and there is no intention in the use of such terms and expression of excluding any equivalents of the features shown and described or portions thereof, it being recognized that various modifications are possible within the scope of the invention.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for alleviating benign prostate hyperplasia (BPH) in a subject in need thereof comprising administering to said subject an omega-3 fatty acid free composition consisting essentially of resveratrol, or an alkyl, alkenyl, or alkynyl substituted resveratrol, in an amount sufficient to alleviate BPH.

2. The method of claim 1, wherein said composition contains resveratrol.

3. The method of claim 1, wherein said composition contains at least one of:

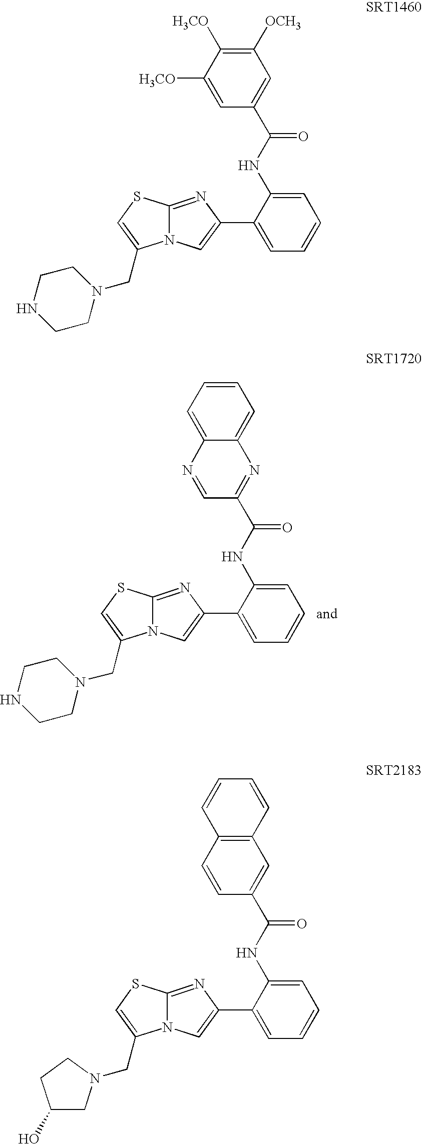

4. The method of claim 1, wherein said composition contains 3,5,4′-trimethoxystilbene or 3,5′-dimethoxy-4-stilbenol.

5. The method of claim 1, wherein said composition is administered orally, intravenously, intramuscularly, transdermally, transepidermally, sublingually, or rectally.

6. The method of claim 1, wherein said composition is administered orally, in the form of a dietary supplement, a tablet, a capsule or a liquid.

7. The method of claim 1, wherein said composition is administered at a dose of from about 10 mg to about 10 g per day of resveratrol or substituted resveratrol.

8. The method of claim 7, wherein said resveratrol or substituted resveratrol is administered at a dose of from about 25 mg to about 7 g per day.

9. The method of claim 8, wherein said resveratrol or substituted resveratrol is administered at a dose of from about 50 mg to about 5 g per day.

10. The method of claim 1, wherein said resveratrol or substituted resveratrol is administered in an amount sufficient to achieve a stable serum level of at least about 10 ng/ml of trans resveratrol.

11. The method of claim 1, comprising administering said resveratrol probiotically.

12. The method of claim 1, wherein said subject is a mammal.

13. The method of claim 12, wherein said mammal is a human.
